(19) United States
(12) Patent Application Publication
(10) Pub. No.: US 2010/0100064 A1
(43) Pub. Date: Apr. 22, 2010

(54) OSTOMY DEVICES WITH MUCOADHESIVES

(75) Inventor: Mahesh Sambasivam, Skillman, NJ (US)

Correspondence Address: CONVATEC INC., 100 HEADQUARTERS PARK DRIVE, SKILLMAN, NJ, 08558, US

(73) Assignee: CONVATEC TECHNOLOGIES INC., Reno, NV (US)

(21) Appl. No.: 12/527,743

(22) PCT Filed: Mar. 6, 2008
(86) PCT No.: PCT/US08/56050

§ 371 Date: Aug. 19, 2009

Related U.S. Application Data
(60) Provisional application No. 60/893,390, filed on Mar. 7, 2007.

(51) Int. Cl. A61F 5/448

(52) U.S. Cl. 604/344

(57) ABSTRACT

The present invention provides a biocompatible adhesive for securely adhering ostomy appliances simultaneously to the body and the stoma. The ostomy appliance is comprised of an adhesive component and a body waste collector component, wherein the adhesive component includes a mucoadhesive component. The mucoadhesive component comprises a polymer with functional groups that provide adhesion to skin and stoma.

### BACKGROUND OF THE INVENTION

There are several medical conditions, such as an ostomy, that require attachment of an appliance to the body to collect waste matter. Ostomy appliances are typically attached to the body via a pressure sensitive adhesive. An ostomy appliance typically includes a wafer containing a pressure sensitive adhesive and a receiving pouch or bag to collect and contain body waste. When the pouch is removably attached to the wafer via a coupling mechanism, it is referred to as a two-piece device. When the wafer and the pouch are permanently attached to each other, the device is referred to as a one-piece device.

The ability of the wafer to adhere to the body is primarily determined by the pressure sensitive adhesive. Unlike other devices that are secured to the body via a pressure sensitive adhesive with minimal or no load on the adhesive, in an ostomy device or appliance, the adhesive has to not only securely hold the device against the body but also be able to bear the weight of the pouch when it is filled with body waste. Prior to application of the appliance, the user trims the opening of the adhesive portion of the one-piece or two-piece appliance to fit around the stoma.

The pressure sensitive adhesives used in ostomy appliances are typically based on hydrocolloids. The hydrocolloids are mixed into a matrix of a pressure sensitive adhesive composition wherein the pressure sensitive adhesive composition provides adhesion to the skin under dry conditions, and the hydrocolloids provide adhesion to the skin under wet/moist conditions.

A significant challenge in adhesion for an ostomy application is the maintenance of sufficient adhesion under wet conditions. The adhesion provided by the hydrocolloids of the conventional ostomy adhesives is not sufficient to maintain a secure adhesion to the body. When the adhesive portion close to the stoma is exposed to fecal matter, the adhesion may be compromised, and the appliance can leak causing embarrassment to the user. If this happens, frequently, the peristomal skin health is also affected which may have a serious impact to the user.

Accordingly, it is desirable to have an appliance that prevents leakage of intestinal effluent in the peristomal area by securely adhering to the body, and directing the effluent into the collection bag.

It is an objective of the present invention to provide a biocompatible adhesive for securely adhering ostomy appliances simultaneously to the body and the stoma. Mucoadhesives are known primarily for use in drug delivery to the buccal cavity or the gastrointestinal tract. Prior to the present invention, it has not been considered for use in an ostomy appliance to provide adhesion simultaneously to the body and the stoma under dry and wet environments.

The mucoadhesives of the present invention are based on polymers with functional groups selected from a group consisting of caprolactams, glycols, amines, alderhydes, hydroxyls, maleimides, thiols, acids and their salts, iminothiolanes, thioalkylamidines, catechols, amino acids and their salts, dihydroxy substituted aromatic groups, and combinations thereof.

U.S. Pat. No. 7,138,458 B2 discloses the preparation of a two-phase, bioadhesive composition comprising an admixture of a crosslinkable hydrophobic composition and a hydrophilic composition.

WO2006092799 A1 discloses a mucoadhesive polymer with a ligand attached to the polymer backbone to interact with toxins.

U.S. Patent Publication No. 2006/0054276 A1 discloses a composition for attaching two surfaces together or coating a surface consisting of an acidic aqueous solution of a polyphenolic protein derived from a byssus-forming mussel.

WO2005117844 A3 discloses a nanocomposite drug delivery system based on chitosan and a network of colloidal nanoporous nanoparticles.

WO2005056708A2 discloses polymers comprising a polymeric backbone and a side chain or side group containing an aromatic group substituted with one or more hydroxyl groups such as catechol for adhesion to biological surfaces to deliver a therapeutic agent.

U.S. Patent Publication No. 20050224175 discloses a composition consisting of an aqueous solution of a polyphenolic protein derived from byssus-forming mussel and mixing with a non-enzymatic oxidizing periodate ions.

### DETAILED DESCRIPTION OF THE INVENTION

It is an objective of the present invention to provide a biocompatible adhesive for securely adhering ostomy appliances simultaneously to the body and the stoma. In one embodiment, the mucoadhesive is part of the appliance prior to use. In another embodiment, the mucoadhesive is used as a separate component to coat the stomal and persitomal area prior to attachment of the appliance. When the mucoadhesive is part of the appliance, it is continuously or discontinuously distributed in the whole wafer and/or around the central area of the wafer contacting the stoma. When the mucoadhesive is a separate component, it can be delivered to the stomal and persitomal area in the form of a liquid, foam, gel, film, paste, sheet, powder, or a combination thereof.

The mucoadhesive of the present invention comprises a polymer with functional groups that provide adhesion to skin and stoma. The functional groups are selected from a group consisting of thiols, acids and their salts, iminothiolanes, thioalkylamidines, catechols, amino acids, dihydroxy substituted aromatic groups, and combinations thereof. The polymer is a biocompatible polymer made from natural or synthetic polymer selected from a group consisting of polyacrylates, polyakylmethacrylates, polyphenylmethacrylate, polyanhydrides, styrenic block copolymers, polyamides, polyesters, polyvinyl ethers, polyvinyl esters, sulfonated polymers, polyolefins, silicones, polyvinylpyrrolidones, polyvinylacetate and its copolymers, polyvinyl acohol, polyurethanes, polyethers, copolymers of maleic anhydride, polysaccharides, polypeptides, gelatin, alginates, gums, starch, chitosan, pectin, and combinations thereof.

The mucoadhesive may further contain other components such as hydrophobic polymers, hydrophilic polymers, amphiphilic polymers, tackifiers, resins, plasticizers, hydrocolloids, inorganic and organic particulate fillers, antioxidants, and combinations thereof.

In another embodiment, an ostomy appliance has an internal and/or external portion to the stoma and a mucoadhesive component coating the portion of the appliance for adhering the coated portion at least to the stoma and/or the persitomal area. Examples of such appliances are an irrigation device and a continent ostomy port device.

### Example 1

A pressure sensitive adhesive composition is prepared by mixing about 20 grams of a copolymer of N-methacrylated L-3,4-dihydroxyphenylalanine (DOPA) and polyethylene glycol diacrylate such as those disclosed by Messersmith et al., J. Biomater. Sci. Polymer Edn, Vol. 15, No. 4, pp. 449-464 (2004), with 40 grams of polyisobutylene L100, and 40 grams of sodium carboxymethyl cellulose. The mucoadhesion of this composition is expected to provide desirable adhesion simultaneously to the stoma and peristomal area.

### Example 2

A pressure sensitive adhesive composition is prepared by mixing about 20 grams of a thiolated chitosan polymer such as those disclosed by Bernkop-Schnurch et al., Biomaterials 26 (2005) 819-826, with 40 grams of polyisobutylene L100, and 40 grams of sodium carboxymethyl cellulose. The mucoadhesion of this composition is expected to provide desirable adhesion simultaneously to the stoma and peristomal area.

I claim:

1. An ostomy appliance comprising an adhesive component for attaching the appliance to the peristomal area and/or stoma, and a body waste collector component, wherein the adhesive component includes a mucoadhesive component.

2. The ostomy appliance according to claim 1, wherein the mucoadhesive component comprises a polymer containing functional groups selected from a group consisting of caprolactams, glycols, amines, alderhydes, hydroxyls, maleimides, thiols, acids and their salts, iminothiolanes, thioalkylamidines, catechols, amino acids, dihydroxy substituted aromatic groups, and combinations thereof.

3. The ostomy appliance according to claim 2, wherein the polymer is a biocompatible polymer of synthetic or natural origin selected from a group consisting of polyacrylates, polyakylmethacrylates, polyphenylmethacrylate, polyanhydrides, styrenic block copolymers, polyamides, polyesters, polyvinyl ethers, polyvinyl esters, sulfonated polymers, polyolefins, silicones, polyvinylpyrrolidones, polyvinylacetate and its copolymers, polyvinyl acohol, polyurethanes, polyethers, copolymers of maleic anhydride, polysaccharides, polypeptides, gelatin, alginates, gums, starch, chitosan, pectin, and combinations thereof.

4. The ostomy appliance according to claim 1, wherein the mucoadhesive further contains other components selected from the group consisting of hydrophobic polymers, hydrophilic polymers, amphiphilic polymers, tackifiers, resins, plasticizers, hydrocolloids, inorganic and organic particulate fillers, antioxidants, and combinations thereof.

5. The ostomy appliance according to claim 1, wherein the mucoadhesive component is a pressure sensitive adhesive.

6. The ostomy appliance according to claim 1, wherein the mucoadhesive component is a non-pressure sensitive adhesive.

7. An ostomy appliance comprising an adhesive component, a body waste collector component, and a separate mucoadhesive component to coat the adhesive component and/or the peristomal area and stoma to secure the appliance to the peristomal area and stoma.

8. The ostomy appliance according to claim 7, wherein the mucoadhesive component comprises a polymer containing functional groups selected from a group consisting of thiols, acids and their salts, iminothiolanes, thioalkylamidines, catechols, amino acids, dihydroxy substituted aromatic groups, and combinations thereof.

9. The ostomy appliance according to claim 7, wherein the mucoadhesive component is a pressure sensitive adhesive.

10. The ostomy appliance according to claim 7, wherein the mucoadhesive component is a non-pressure sensitive adhesive.

11. An ostomy appliance having an internal and/or external portion to the stoma and a mucoadhesive component coating the portion of the appliance for adhering said coated portion at least to the stoma and/or the persitomal area.
